SEQUENCE LISTING (1 sequence)

SEQ ID NO: 1 - protein, 17 aa, Artificial Sequence
  Description of Artificial Sequence Synthetic peptide
IDQQVLSRIKLEIKRCL

(19) United States
(12) Patent Application Publication
(10) Pub. No.: US 2020/0061150 A1
(43) Pub. Date: Feb. 27, 2020

(54) METHOD OF INDUCING SELECTIVE PROSTATE GLANDULAR PHARMACO-ABLATION WITH SPARING OF NERVES AND PRESERVATION OF SEXUAL FUNCTION

(71) Applicant: Nymox Corporation, Nassau (BS)

(72) Inventor: Paul Averback, Nassau (BS)

(21) Appl. No.: 16/110,549

(22) Filed: Aug. 23, 2018

(51) Int. Cl. A61K 38/10; A61P 15/08

(52) U.S. Cl. CPC A61K 38/10; A61P 15/08

(57) ABSTRACT

Disclosed are methods of selective glandular pharmaco-ablation using compositions containing compounds based on small peptides and a pharmaceutically acceptable carrier. The methods of selectively destroying prostate gland overgrowth substantially or completely preserve key nerve, stromal, vascular, connective tissue, urethral musculature, and structural elements in intimate structural proximity to the foci of ablation.

### BACKGROUND

### 1. Field of the Embodiments

The embodiments include methods of selective prostate glandular pharmaco-ablation using compositions containing compounds based on small peptides and a pharmaceutically acceptable carrier. More particularly, the embodiments include methods of selectively destroying prostate gland overgrowth while preserving key nerve, stromal, vascular, connective tissue, urethral musculature, and structural elements in intimate structural proximity to the foci of ablation.

### 2. Description of Related Art

The essence of many medical treatments and procedures involves the removal or destruction of harmful or unwanted tissue. Examples of such treatments include the surgical removal of cancerous or pre-cancerous growths, the destruction of metastatic tumors through chemotherapy, and the reduction of glandular (e.g. prostate) hyperplasia. Other examples include the removal of unwanted facial hair, the removal of warts, and the removal of unwanted fatty tissue.

Benign prostatic hyperplasia (BPH) is common in older men, with symptoms that impact quality of life, including interference with activities and perception of well-being. BPH can be progressive, with risk of urinary retention, infections, bladder calculi, and renal failure. Although many men with mild to moderate symptoms do well without intervention, bothersome symptoms and complications can progress in others, leading to medical therapy or surgery.

The United States and Europe have established guidelines to assist physicians in the treatment of LUTS, BPH, and LUTS/BPH. Oelke M, et al., European Association of Urology, Eur. Urol. 2013 July; 64(1):118-40. The guidelines discuss treatment options varying from watchful waiting (WW), for men presenting with symptoms but are not bothered enough to need medication or surgical intervention, to drug treatments, to surgical intervention. Drug treatment guidelines have included the use of alpha-blockers (alpha-adrenergic antagonists), 5-alpha-reductase inhibitors (5ARIs), antimuscarinics (anticholinergics), a PDE5 inhibitor (tadalafil), combination therapies, and vasopressin analogues. The use of combination therapies such as an alpha-blocker with a 5ARI or antimuscarinic also have been recommended.

Prostate surgery such as transurethral resection of the prostate is indicated in men with absolute indications or drug treatment-resistant BPH, LUTS, or acute urinary retention (AUR). Indications for surgery include severe conditions such as urinary retention, gross hematuria, urinary tract infection, and bladder stones. Minimally invasive treatments include transurethral microwave therapy and transurethral needle therapy. An alternative to catheterization for men unfit for surgery include prostate stents. Despite the various available treatment options, there remain unmet medical needs for effective and safe agents to treat these bothersome symptoms, some of which may be caused by prostate enlargement, which can lead to more serious problems such as chronic urinary tract infections, incontinence, acute and chronic urinary retention, and renal failure.

There is a need for an effective composition that will destroy and hence either facilitate the removal of or inhibit the further growth of harmful or unwanted cells and tissue but will have mainly local effects and minimal or absent systemic toxicity. There also is a need to reduce the need for invasive surgical intervention, even after treatment with an effective composition.

Some agents known to have the ability to destroy and hence either facilitate the removal of or inhibit the further growth of harmful or unwanted cells and tissue are disclosed in U.S. patent application Ser. No. 14/808,713, filed Jul. 24, 2015, entitled: METHODS OF REDUCING THE NEED FOR SURGERY IN PATIENTS SUFFERING FROM BENIGN PROSTATIC HYPERPLASIA; U.S. patent application Ser. No. 14/606,683, filed Jan. 27, 2015, entitled: METHOD OF TREATING DISORDERS REQUIRING DESTRUCTION OR REMOVAL OF CELLS, U.S. application Ser. No. 14/738,551, filed Jun. 12, 2015, entitled: COMBINATION COMPOSITIONS FOR TREATING DISORDERS REQUIRING REMOVAL OR DESTRUCTION OF UNWANTED CELLULAR PROLIFERATIONS, U.S. patent application Publication Nos. 2007/0237780 (now abandoned); 2003/0054990 (now U.S. Pat. No. 7,172,893); 2003/0096350 (now U.S. Pat. No. 6,924,266); 2003/0096756 (now U.S. Pat. No. 7,192,929); 2003/0109437 (now U.S. Pat. No. 7,241,738); 2003/0166569 (now U.S. Pat. No. 7,317,077); 2005/0032704 (now U.S. Pat. No. 7,408,021); and 2015/0148303 (now U.S. Pat. No. 9,243,035), the disclosures of each of which are incorporated by reference herein in their entirety.

Benign overgrowths of tissue are abnormalities in which it is desirable to remove cells from an organism. Benign tumors are cellular proliferations that do not metastasize throughout the body but do cause disease symptoms. Such tumors can be lethal if they are located in inaccessible areas in organs such as the brain. There are benign tumors of organs including lung, brain, skin, pituitary, thyroid, adrenal cortex and medulla, ovary, uterus, testis, connective tissue, muscle, intestines, ear, nose, throat, tonsils, mouth, liver, gall bladder, pancreas, prostate, heart, and other organs.

Surgery often is the first step in the treatment of cancer. The objective of surgery varies. Sometimes it is used to remove as much of the evident tumor as possible, or at least to “debulk” it (remove the major bulk(s) of tumor so that there is less that needs to be treated by other means). Depending on the cancer type and location, surgery may also provide some symptomatic relief to the patient. For instance, if a surgeon can remove a large portion of an expanding brain tumor, the pressure inside the skull will decrease, leading to improvement in the patient's symptoms.

Not all tumors, however, are amenable to surgery. Some may be located in parts of the body that make them impossible to completely remove. Examples of these would be tumors in the brainstem (a part of the brain that controls breathing) or a tumor which has grown in and around a major blood vessel. In these cases, the role of surgery is limited due to the high risk associated with tumor removal.

In some cases, surgery is not used to debulk tumor tissue because it is simply not necessary. An example is Hodgkin's lymphoma, a cancer of the lymph nodes that responds very well to combinations of chemotherapy and radiation therapy. In Hodgkin's lymphoma, surgery is rarely needed to achieve cure, but almost always used to establish a diagnosis.

Chemotherapy is another common form of cancer treatment. Essentially, it involves the use of medications (usually given by mouth or injection) which specifically attack rapidly dividing cells (such as those found in a tumor) throughout the body. This makes chemotherapy useful in treating cancers that have already metastasized, as well as tumors that have a high chance of spreading through the blood and lymphatic systems but are not evident beyond the primary tumor. Chemotherapy may also be used to enhance the response of localized tumors to surgery and radiation therapy. This is the case, for example, for some cancers of the head and neck.

Unfortunately, other cells in the human body that also normally divide rapidly (such as the lining of the stomach and hair) also are affected by chemotherapy. For this reason, many chemotherapy agents induce undesirable side effects such as nausea, vomiting, anemia, hair loss or other symptoms. These side effects are temporary, and there exist medications that can help alleviate many of these side effects. As our knowledge has continued to grow, researchers have devised newer chemotherapeutic agents that are not only better at killing cancer cells, but that also have fewer side effects for the patient.

Chemotherapy is administered to patients in a variety of ways. Some include pills and others are administered by an intravenous or other injection. For injectable chemotherapy, a patient goes to the doctor's office or hospital for treatment. Other chemotherapeutic agents require continuous infusion into the bloodstream, 24 hours a day. For these types of chemotherapy, a minor surgical procedure is performed to implant a small pump worn by the patient. The pump then slowly administers the medication. In many cases, a permanent port is placed in a patient's vein to eliminate the requirement of repeated needle sticks.

Benign tumors and malformations also can be treated by a variety of methods including surgery, radiotherapy, drug therapy, thermal or electric ablation, cryotherapy, and others. Although benign tumors do not metastasize, they can grow large and they can recur. Surgical extirpation of benign tumors has all the difficulties and side effects of surgery in general and oftentimes must be repeatedly performed for some benign tumors, such as for pituitary adenomas, meningeomas of the brain, prostatic hyperplasia, and others. In addition, some patients who receive non-surgical treatment to ameliorate the symptoms caused by benign tumors, still require subsequent invasive surgical intervention. Lepor, “Medical Treatment of Benign Prostatic Hyperplasia,” Reviews in Urology, Vol. 13, No. 1, pp. 20-33 (2011), discloses a variety of studies of the efficacy of drug therapies in treating BPH, and the need for subsequent invasive surgical treatment.

In all or most of these instances, there is a need for treatments that can remove, destroy, or ameliorate the unwanted conditions associated with BPH, LUTS, or AUR without the risks and side effects of conventional therapies, or treatments that can remove, destroy, or ameliorate the unwanted condition with more precision.

Removal of tissue overgrowth is commonly required in many prostatic disease conditions. Prostate cancers are removed by surgical means and/or radiation, chemotherapy, or focal treatment. In BPH, when symptoms are severe the enlarged transition zone glandular overgrowth may require ablation by surgical resection, or by laser, microwave, high intensity ultrasound, thermal needle placement, steam, or other methods of transition zone tissue destruction.

In ablation methods that destroy tissue, the zones of tissue destruction are microscopically non-selective, which can be attributed to the non-selective forces (high energy transduction, radiation) which causes the necrosis. Therefore, there is a need for treatments that can effectively produce tissue destruction that is structurally selective at the microscopic (histological) level, in order to avoid undesirable toxicities and irreparable damage to key adjacent structures. For example, transurethral resection, high energy laser extirpations, and other methods frequently damage prostatic nerve, stromal, vascular, and connective tissue, and urethral musculature, with frequent consequent occurrence of permanent ejaculatory disorders and impotence, and less often other undesirable events, such as incontinence.

Throughout this description, including the foregoing description of related art, any and all publicly available documents described herein, including any and all U.S. patent published patent applications, are specifically incorporated by reference herein in their entirety. The foregoing description of related art is not intended in any way as an admission that any of the documents described therein, including pending U.S. patent applications, are prior art to the present disclosure. Moreover, the description herein of any disadvantages associated with the described products, methods, and/or apparatus, is not intended to limit the embodiments. Indeed, aspects of the embodiments may include certain features of the described products, methods, and/or apparatus without suffering from their described disadvantages.

### SUMMARY OF THE EMBODIMENTS

There remains a need in the art for new, less toxic, and less frequent (e.g., avoiding the need to take medications daily or weekly) treatments for selectively inducing apoptosis in prostatic tissue overgrowths while preserving the prostatic nerve, stromal, vascular, and connective tissue, and urethral musculature. Prior treatments for prostatic tissue overgrowths were either focused to select loci in the prostate gland, resulting in relatively incomplete treatment, or if less focused, caused irreparable damage to adjacent tissue, nerves and musculature. In addition, prior treatments for prostatic tissue overgrowths that employed injection of peptides, used a small dose of peptide (about 0.25 mg/ml) focused in a limited area of the prostate. Accordingly, there also remains a need for treatments to remove prostatic tissue overgrowths that can be administered more generally to the tissue, while preserving adjacent tissue such as prostatic nerve, stromal, vascular, and connective tissue, and urethral musculature. The embodiments described herein satisfy these needs.

This disclosure is premised in part on the discovery that certain peptides, including a specific peptide described by the amino acid sequence Ile-Asp-Gln-Gln-Val-Leu-Ser-Arg-Ile-Lys-Leu-Glu-Ile-Lys-Arg-Cys-Leu, (Fexapotide Triflutate or “FT”) are capable of selectively inducing apoptosis in prostatic tissue overgrowths while preserving the prostatic nerve, stromal, vascular, and connective tissue, and urethral musculature.

This disclosure also is premised in part on the discovery that the use of FT either alone or in combination with an additional active agent treats and/or kills unwanted cellular proliferations in mammals without significant known molecular adverse events from interactions with other tissues. While FT has previously been used to destroy unwanted cellular proliferations, it was not known to be selective in inducing apoptosis. Consequently, prior treatments with FT typically involved direct injection at the loci of unwanted cellular proliferations to avoid destruction of healthy cells such as nerve, stromal, vascular, and connective tissue, and urethral musculature. The present inventor unexpectedly discovered that FT induced selective prostate glandular pharmaco-ablation, and consequently, the compositions can be administered more generally and preferably less invasively, and can be administered in significantly higher dosages. In accordance with an embodiment, there is provided a method of inducing selective prostate glandular pharmaco-ablation by administering FT in an amount sufficient to treat a substantial portion of the prostate gland.

The compositions can be administered intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transrectally, transperitoneally, transdermally, via an aerosol, infusion, bolus injection, implantation device, sustained release system etc. Alternatively, the FT peptide can be expressed in vivo by administering a gene that expresses the peptide, by administering a vaccine that induces such production or by introducing cells, bacteria or viruses that express the peptide in vivo, because of genetic modification or otherwise.

In another embodiment, administering a composition comprising FT, either alone or in combination with at least one additional active agent capable of treating and/or killing unwanted cellular proliferations in mammals, reduces the prostate volume by up to 10% when compared to administering a control composition that does not contain FT.

Both the foregoing general description and the following detailed description are exemplary and explanatory and are intended to provide further explanation of the embodiments as claimed. Other objects, advantages, and features will be readily apparent to those skilled in the art from the following detailed description of the embodiments.

### DESCRIPTION OF THE DRAWINGS

FIG. 1a is a graph showing the mean prostate volume for mammals administered FT vs. controls over the period of 24 hours to 7 days.

FIG. 1b is a graph showing the mean prostate volume for mammals administered FT vs. controls over the period of 0 to 12 months.

### DETAILED DESCRIPTION OF THE PREFERRED EMBODIMENTS

Before the embodiments are described, it is understood that this invention is not limited to the particular methodology, protocols, cell lines, vectors, and reagents described, as these may vary. It also is to be understood that the terminology used herein is for the purpose of describing particular embodiments only, and is not intended to limit the scope of the present embodiments which will be limited only by the appended claims.

Terms and phrases used herein are defined as set forth below unless otherwise specified. Throughout this description, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Amino acids and amino acid residues described herein may be referred to according to the accepted one or three-letter code provided in the table below.

TABLE 1

| Three-Letter Amino | One-Letter | |
|---|---|---|
| Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Fexapotide Triflutate (“FT”), as it is used herein, denotes a 17-mer peptide having the amino acid sequence: Ile-Asp-Gln-Gln-Val-Leu-Ser-Arg-Ile-Lys-Leu-Glu-Ile-Lys-Arg-Cys-Leu (SEQ ID NO. 1). FT is disclosed in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031. The disclosures of these patents and published applications are incorporated by reference herein in their entirety.

FT is represented by:

SEQ ID NO.1:
IDQQVLSRIKLEIKRCL
or
Ile-Asp-Gln-Gln-Val-Leu-Ser-Arg-Ile-Lys-Leu-Glu-
Ile-Lys-Arg-Cys-Leu.

The term “fragment” refers to a protein or polypeptide that consists of a continuous subsequence of the amino acid sequence of a protein or peptide and includes naturally occurring fragments such as splice variants and fragments resulting from naturally occurring in vivo protease activity. Such a fragment may be truncated at the amino terminus, the carboxy terminus, and/or internally (such as by natural splicing). Such fragments may be prepared with or without an amino terminal methionine. The term “fragment” includes fragments, whether identical or different, from the same protein or peptide, with a contiguous amino acid sequence in common or not, joined together, either directly or through a linker. A person having ordinary skill in the art will be capable of selecting a suitable fragment for use in the embodiments without undue experimentation using the guidelines and procedures outlined herein.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in Table 2 below.

TABLE 2
Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

TABLE 3
Table 3 sets out another scheme of amino acid substitution:

| Original Residue | Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | eu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. For example, a cysteine residue may be added at both the amino and carboxy terminals of FT in order to allow the cyclisation of the peptide by the formation of a di-sulphide bond. The term “variant” also encompasses polypeptides that have the amino acid sequence of FT with at least one and up to 25 or more additional amino acids flanking either the 3′ or 5′ end of the peptide.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type proteins or FT. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide. Also, a given protein or polypeptide may contain many types of modifications. Modifications can occur anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2nd Ed., T. E. Creighton, W. H. Freeman and Company, New York (1993) and Wold, F., “Posttranslational Protein Modifications: Perspectives and Prospects,” pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., “Protein Synthesis: Posttranslational Modifications and Aging,” Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

The term “homologue” refers to a protein that is at least 60 percent identical in its amino acid sequence of FT as determined by standard methods that are commonly used to compare the similarity in position of the amino acids of two polypeptides. The degree of similarity or identity between two proteins can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo H. and Lipman, D., SIAM, J. Applied Math., 48:1073 (1988). Preferred methods to determine identity are designed to give the largest match between the sequences tested. Methods to determine identity and similarity are codified in publicly available computer programs.

Preferred computer program methods useful in determining the identity and similarity between two sequences include, but are not limited to, the GCG program package (Devereux, J., et al., Nucleic Acids Research, 12(1): 387 (1984)), BLASTP, BLASTN, and FASTA, Atschul, S. F. et al., J. Molec. Biol., 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol., 215: 403-410 (1990). By way of example, using a computer algorithm such as GAP (Genetic Computer Group, University of Wisconsin, Madison, Wis.), the two proteins or polypeptides for which the percent sequence identity is to be determined are aligned for optimal matching of their respective amino acids (the “matched span”, as determined by the algorithm).

A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al. in: Atlas of Protein Sequence and Structure, vol. 5, supp.3 for the PAM250 comparison matrix; see Henikoff et al., Proc. Natl. Acad. Sci USA, 89:10915-10919 for the BLOSUM 62 comparison matrix) also may be used by the algorithm. The percent identity then is calculated by the algorithm. Homologues will typically have one or more amino acid substitutions, deletions, and/or insertions as compared with the comparison protein or peptide, as the case may be.

The term “fusion protein” refers to a protein where one or more peptides are recombinantly fused or chemically conjugated (including covalently and non-covalently) to a protein such as (but not limited to) an antibody or antibody fragment like an Fab fragment or short chain Fv. The term “fusion protein” also refers to multimers (i.e. dimers, trimers, tetramers and higher multimers) of peptides. Such multimers comprise homomeric multimers comprising one peptide, heteromeric multimers comprising more than one peptide, and heteromeric multimers comprising at least one peptide and at least one other protein. Such multimers may be the result of hydrophobic, hydrophilic, ionic and/or covalent associations, bonds or links, may be formed by cross-links using linker molecules or may be linked indirectly by, for example, liposome formation.

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to the embodiments provide a spatial arrangement of reactive chemical moieties that closely resemble the three-dimensional arrangement of active groups in the peptide on which the peptide mimetic is based. As a result of this similar active-site geometry, the peptide mimetic has effects on biological systems that are similar to the biological activity of the peptide.

The peptide mimetics of the embodiments are preferably substantially similar in both three-dimensional shape and biological activity to the peptides described herein. Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala.sup.1-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the peptide by pseudopeptide bonds that confer resistance to proteolysis.

A number of pseudopeptide bonds have been described that in general do not affect peptide structure and biological activity. One example of this approach is to substitute retro-inverso pseudopeptide bonds (“Biologically active retroinverso analogues of thymopentin”, Sisto A. et al in Rivier, J. E. and Marshall, G. R. (eds) “Peptides, Chemistry, Structure and Biology”, Escom, Leiden (1990), pp. 722-773) and Dalpozzo, et al. (1993), Int. J. Peptide Protein Res., 41:561-566, incorporated herein by reference). According to this modification, the amino acid sequences of the peptides may be identical to the sequences of an peptide described above, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. Further modifications also can be made by replacing chemical groups of the amino acids with other chemical groups of similar structure. Another suitable pseudopeptide bond that is known to enhance stability to enzymatic cleavage with no or little loss of biological activity is the reduced isostere pseudopeptide bond (Couder, et al. (1993), Int. J. Peptide Protein Res., 41:181-184, incorporated herein by reference in its entirety).

Thus, the amino acid sequences of these peptides may be otherwise identical to the sequence of FT, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above). Other examples include the introduction of ketomethylene or methylsulfide bonds to replace peptide bonds.

Peptoid derivatives of peptides represent another class of peptide mimetics that retain the important structural determinants for biological activity, yet eliminate the peptide bonds, thereby conferring resistance to proteolysis (Simon, et al., 1992, Proc. Natl. Acad. Sci. USA, 89:9367-9371, incorporated herein by reference in its entirety). Peptoids are oligomers of N-substituted glycines. A number of N-alkyl groups have been described, each corresponding to the side chain of a natural amino acid (Simon, et al. (1992), cited above). Some or all of the amino acids of the peptides may be replaced with the N-substituted glycine corresponding to the replaced amino acid.

The term “peptide mimetic” or “mimetic” also includes reverse-D peptides and enantiomers as defined below.

The term “reverse-D peptide” refers to a biologically active protein or peptide consisting of D-amino acids arranged in a reverse order as compared to the L-amino acid sequence of an peptide. Thus, the carboxy terminal residue of an L-amino acid peptide becomes the amino terminal for the D-amino acid peptide and so forth. For example, the peptide, ETESH, becomes H_{d}S_{d}E_{d}T_{d}E_{d}, where E_{d}, H_{d}, S_{d}, and T_{d} are the D-amino acids corresponding to the L-amino acids, E, H, S, and T respectively.

The term “enantiomer” refers to a biologically active protein or peptide where one or more the L-amino acid residues in the amino acid sequence of an peptide is replaced with the corresponding D-amino acid residue(s).

A “composition” as used herein, refers broadly to any composition containing FT and, optionally an additional active agent. The composition may comprise a dry formulation, an aqueous solution, or a sterile composition. Compositions comprising FT may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

In an embodiment in which an additional active agent is used together with FT, the expression “active agent” is used to denote any agent that provides a therapeutic effect to a subject in need, and preferably is an agent capable of removing unwanted cellular proliferations and/or tissue growth. Suitable active agents may include, but are not limited to: (i) anti-cancer active agents (such as alkylating agents, topoisomerase I inhibitors, topoisomerase II inhibitors, RNA/DNA antimetabolites, and antimitotic agents); (ii) active agents for treating benign growths such as anti-acne and anti-wart active agents; (iii) antiandrogen compounds, (cyproterone acetate (1α, 2β-methylene-6-chloro-17α-acetoxy-6-dehydroprogesterone) Tamoxifen, aromatase inhibitors); (iv). alpha1-adrenergic receptor blockers (tamsulosin, terazosin, doxazosin, prazosin, bunazosin, indoramin, alfulzosin, silodosin); (v) 5α-reductase inhibitors (finasteride, dutasteride); (vi) phosphodiesterase type 5 (PDE5) inhibitors (tadalafil) and combinations thereof.

While not intending on being bound by any particular theory or operation, the inventor unexpectedly discovered that administration of FT, alone or in combination with another active agent, led to apoptosis in prostate glandular epithelium and to widespread but selective gland epithelial cell loss and atrophy. The inventor further discovered that the selective gland epithelial cell loss and atrophy was achieved while preserving adjacent tissue such as prostatic nerve, stromal, vascular, and connective tissue, and urethral musculature.

Mammals treated with the compositions described herein exhibited a prostate gland shrinkage per single dose, when compared to administering a control composition that does not contain FT, by an amount within the range of from about 15 to about 75%, or from about 25 to about 50%, or from about 33 to about 48%.

The embodiments include a method of treating a mammal suffering from prostate tissue overgrowth, comprising administering once or more than once FT to the mammal, either alone or in combination with administration of an additional active agent. The method includes, but is not limited to, administering composition comprising FT intramuscularly, orally, intravenously, intraperitoneally, intracerebrally (intraparenchymally), intracerebroyentricularly, intralesionally, intraocularly, intraarterially, intrathecally, intratumorally, Intranasally, topically, transdermally, subcutaneously, intradermally, transrectally, transperitoneally, either alone or conjugated to a carrier. The composition comprising FT, either alone or in combination with an additional active agent, can be administered in an amount sufficient to treat a substantial portion of the prostate gland. The term “substantial” is intended to mean most or all of the prostate gland, and can include more than 75% of the prostate gland, or more than 80%, or more than 85%, or more than 90%, or more than 95%, or more than 98%, or the entire prostate gland. The composition can be administered in such a manner by administering a higher dose in one area of the gland, and/or by administering the composition at more than one, or more than two, or up to 10 different foci of the prostate gland, thereby resulting in a significantly higher dose than previously administered. When administered to substantially the entire gland in an increased dosage amount, the compositions comprising FT may further be useful in preventing smaller cancers by having more access to the entire gland. For example, administration of the compositions described herein can result in a remarkably reduced incidence of prostate cancer (1% where typically the incidence is about 20%).

Any mammal can benefit from use of the invention, including humans, mice, rabbits, dogs, sheep and other livestock, any mammal treated or treatable by a veterinarian, zoo-keeper, or wildlife preserve employee. Preferred mammals are humans, sheep, and dogs. Throughout this description mammals and patients are used interchangeably.

It will be apparent to one of skill in the art that other smaller fragments of FT may be selected such that these peptides will possess the same or similar biological activity. Other fragments of FT may be selected by one skilled in the art such that these peptides will possess the same or similar biological activity. The peptides of the embodiments encompass these other fragments. In general, the peptides of the embodiments have at least 4 amino acids, preferably at least 5 amino acids, and more preferably at least 6 amino acids.

The embodiments also encompass methods of treating mammals (or patients) suffering from prostate tissue overgrowth comprising administering a composition comprising FT that includes two or more FT sequences joined together, together with an additional active agent. To the extent that FT has the desired biological activity, it follows that two or more FT sequences would also possess the desired biological activity.

FT and fragments, variants, derivatives, homologues, fusion proteins and mimetics thereof encompassed by this embodiment can be prepared using methods known to those of skill in the art, such as recombinant DNA technology, protein synthesis and isolation of naturally occurring peptides, proteins, variants, derivatives and homologues thereof. FT and fragments, variants, derivatives, homologues, fusion proteins and mimetics thereof can be prepared from other peptides, proteins, and fragments, variants, derivatives and homologues thereof using methods known to those having skill in the art. Such methods include (but are not limited to) the use of proteases to cleave the peptide, or protein into FT. Any method disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used to prepare the FT peptide described herein.

The present embodiments are directed to methods of treating mammals suffering from BPH, LUTS, AUR, prostate cancer, or other disorders requiring the removal or destruction of cellular overgrowths, whereby the treatment selectively removes glandular tissue with complete or near complete preservation of key nerve, stromal, vascular, connective tissue, urethral musculature, and structural elements in intimate structural proximity to the foci of treatment. Such a method comprises administering to a mammal in need thereof, a therapeutically effective amount of FT, either alone, or in combination with an additional active agent in an amount sufficient to treat a substantial portion of the prostate gland. The mammals in need may be mammals suffering from BPH, LUTS, AUR, or prostate cancer, irrespective of mammals also suffering from benign prostatic hyperplasia.

The additional active agent, if used, can be one or more active agents selected from (i) anti-cancer active agents (such as alkylating agents, topoisomerase I inhibitors, topoisomerase II inhibitors, RNA/DNA antimetabolites, and antimitotic agents); (ii) active agents for treating benign growths such as anti-acne and anti-wart active agents (salicylic acid); (iii) antiandrogen compounds, (cyproterone acetate (1α, 2β-methylene-6-chloro-17α-acetoxy-6-dehydroprogesterone)) Tamoxifen, aromatase inhibitors); (iv) alpha1-adrenergic receptor blockers (tamsulosin, terazosin, doxazosin, prazosin, bunazosin, indoramin, alfuizosin, silodosin); (v) 5α-reductase inhibitors (finasteride, dutasteride); (vi) phosphodiesterase type 5 (PDE5) inhibitors (tadalafil) and combinations thereof. Preferably, the additional active agent is selected from the group consisting of tamsulosin, finasteride, terazosin, doxazosin, prazosin, tadalafil, alfuzosin, silodosin, dutasteride, combinations of dutasteride and tamsulosin, and mixtures and combinations thereof.

Therapeutic compositions described herein may comprise a therapeutically effective amount of FT in admixture with a pharmaceutically acceptable carrier. In some alternative embodiments, the additional active agent can be administered in the same composition with FT, and in other embodiments, the composition comprising FT is administered as an injection, whereas the additional active agent is formulated into an oral medication (gel, capsule, tablet, liquid, etc.). The carrier material may be water for injection, preferably supplemented with other materials common in solutions for administration to mammals. Typically, FT will be administered in the form of a composition comprising the purified FT peptide in conjunction with one or more physiologically acceptable carriers, excipients, or diluents. Neutral buffered saline or saline mixed with serum albumin are exemplary appropriate carriers. Preferably, the product is formulated as a lyophilizate using appropriate excipients (e.g., sucrose). Other standard carriers, diluents, and excipients may be included as desired. Compositions of the embodiments also may comprise buffers known to those having ordinary skill in the art with an appropriate range of pH values, including Tris buffer of about pH 7.0-8.5, or acetate buffer of about pH 4.0-5.5, which may further include sorbitol or a suitable substitute therefor.

Solid dosage forms for oral administration include but are not limited to, capsules, tablets, pills, powders, and granules. In such solid dosage forms, the additional active agent, and/or FT can be admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, alginates, gelatin, polyvinylpyrrolidone, sucrose and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as acetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active compounds, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition can also include adjuvants, such as wetting agents, emulsifying and suspending agents, sweetening, flavoring, and perfuming agents.

Actual dosage levels of active ingredients in the compositions of the embodiments may be varied to obtain an amount of FT and additional active agent that is effective to obtain a desired therapeutic response for a particular composition and method of administration. The selected dosage level therefore depends upon the desired therapeutic effect, the route of administration, the desired duration of treatment, and other factors.

With mammals, including humans, the effective amounts can be administered on the basis of body surface area. The interrelationship of dosages for animals of various sizes, species and humans (based on mg/M² of body surface) is described by E. J. Freireich et al., Cancer Chemother. Rep., 50 (4):219 (1966). Body surface area may be approximately determined from the height and weight of an individual (see e.g., Scientific Tables, Geigy Pharmaceuticals, Ardsley, N.Y. pp. 537-538 (1970)).

The total daily dose of the FT peptide and optional additional active agent administered to a host may be in single or divided doses. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, potency of the administered drug, rates of absorption and excretion, combination with other drugs and the severity of the particular disease being treated. It is preferred that the composition is administered only once as an injection or infusion, or in another preferred embodiment, the composition is administered in more than one location in the gland. In this embodiment, the period of time between administration of the composition may vary anywhere from 2 months to 10 years, or from 8 months to 4 years, or more than about one year (e.g., between 1 and 2 years).

A method of administering a composition comprising FT according to the embodiments includes, but is not limited to, administering the compositions intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transrectally, transperitoneally, transdermally, via an aerosol, infusion, bolus injection, implantation device, sustained release system etc. Any method of administration disclosed in, for example, U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, can be used.

In certain embodiments, the isolated FT peptide can be administered in combination with at least one active agent selected from the group consisting of (1) of an inhibitor of 5α-reductase and/or an antiestrogen, (2) an inhibitor of 5α-reductase and/or an aromatase inhibitor, (3) a 5α-reductase inhibitor and/or a 17β-HSD inhibitor, (4) a 5α-reductase inhibitor, an antiestrogen and an aromatase inhibitor, (5) a 5α-reductase inhibitor, an antiestrogen and a 17-HSD inhibitor, (6) a 5α-reductase inhibitor, an aromatase inhibitor, an antiestrogen and a 17β-HSD inhibitor, (7) a 5α-reductase inhibitor, an antiandrogen and an antiestrogen, (8), a 5α-reductase inhibitor, an antiandrogen and an aromatase inhibitor, (9) a 5α-reductase inhibitor, an antiandrogen and an 17β-HSD inhibitor, (10) a 5α-reductase inhibitor, an antiandrogen, an antiestrogen and an aromatase inhibitor, (11) a 5α-reductase inhibitor, an antiandrogen, an aromatase inhibitor and a 17β-HSD inhibitor, (12) a 5α-reductase inhibitor, an antiandrogen, an aromatase inhibitor, an antiestrogen and a 17β-HSD inhibitor, (13) a 17β-HSD inhibitor and an antiestrogen, (14) a 17-HSD inhibitor and an aromatase inhibitor, (15) a 17-HSD inhibitor, an aromatase inhibitor and an antiestrogen, (16) a 17-HSD inhibitor, an antiandrogen and an antiestrogen, (17) a 17β-HSD inhibitor, an antiandrogen and an aromatase inhibitor, (18) a 17-HSD inhibitor, an antiandrogen, an antiestrogen and an aromatase inhibitor, (19) an antiestrogen and an aromatase inhibitor and (20) an antiestrogen, an aromatase inhibitor, and an antiandrogen, (21) an LHRH agonist or antagonist, an inhibitor of 5α-reductase and an antiestrogen, (22) an LHRH agonist or antagonist, an Inhibitor of 5α-reductase and an aromatase inhibitor, (23) an LHRH agonist or antagonist, a 5a reductase inhibitor and a 17β-HSD inhibitor, (24) an LHRH agonist or antagonist, a 5α-reductase inhibitor, an antiestrogen and an aromatase inhibitor, (25) an LHRH agonist or antagonist, a 5α-reductase inhibitor, an antiestrogen and a 17β-HSD inhibitor, (26) an LHRH agonist or antagonist, a 5α-reductase inhibitor, an aromatase inhibitor, an antiestrogen and a 17β-HSD inhibitor, (27) an LHRH agonist or antagonist, a 5α-reductase inhibitor, an antiandrogen and an antiestrogen, (28), an LHRH agonist or antagonist, a 5α-reductase Inhibitor, an antiandrogen and an aromatase inhibitor, (29) an LHRH agonist or antagonist, a 5α-reductase inhibitor, an antiandrogen and an 17β-HSD inhibitor, (30) an LHRH agonist or antagonist, a 5α-reductase inhibitor, an antiandrogen, an antiestrogen and an aromatase inhibitor, (31) an LHRH agonist or antagonist, a 5α-reductase inhibitor, an antiandrogen, an aromatase inhibitor and a 17β-HSD inhibitor, (32) an LHRH agonist or antagonist, a 5α-reductase inhibitor, an antiandrogen, an aromatase inhibitor, an antiestrogen and a 17β-HSD inhibitor, (33) an LHRH agonist or antagonist, a 17β-HSD inhibitor and an antiestrogen, (34) an LHRH agonist or antagonist, a 17β-HSD inhibitor and an aromatase inhibitor, (35) an LHRH agonist or antagonist, a 17β-HSD inhibitor, an aromatase inhibitor and an antiestrogen, (36) an LHRH agonist or antagonist, a 17β-HSD Inhibitor, an antiandrogen and an antiestrogen, (37) an LHRH agonist or antagonist, a 17β-HSD inhibitor, an antiandrogen and an aromatase inhibitor, (38) an LHRH agonist or antagonist, a 17β-HSD inhibitor, an antiandrogen, an antiestrogen and an aromatase inhibitor, (39) an LHRH agonist or antagonist, an antiestrogen and an aromatase inhibitor and (40) an LHRH agonist or antagonist, an antiestrogen, an aromatase inhibitor, and an antiandrogen.

FT is a new molecular entity which in vitro stimulates caspase pathways (activation of caspases 7, 8, and 10, caspase recruitment domains 6, 11, and 14, and DIABLO), tumor necrosis factor pathways (activation of TNF1, TNFSF6, TNFSF8, TNFSF9, CD70 ligands, and TNFRSF19L, TNFRSF25, TRAF2, TRAF3, TRAF4, TRAF6 receptors), and BCL pathways (activation of BIK, HRK, BCL2L10 and BCL3) in prostate glandular epithelial cells, based on tissue culture genetic array data. FT selectively causes loss of cell membrane integrity, mitochondrial metabolic arrest, depletion of RNA, DNA lysis and aggregation, and cell fragmentation and cell loss. The apoptotic process leads to typical ultrastructural progressive changes of membranous disruption and swelling, progressively deepening nuclear invaginations with eventual membranous bleb formations and cell death and fragmentation into apoptotic bodies. Histologically, typical apoptotic changes with positive immunohistochemical staining of markers for apoptosis are found throughout the injected areas for up to several weeks after treatment.

FT has been extensively tested in patients with BPH and in men with low-grade (T1c) prostate cancer. The compound and placebo controls have been administered by the transrectal route in over 1700 procedures in 9 human clinical trials. In these large long-term clinical trials in men with BPH, FT was administered in a concentration of 0.25 mg/ml (2.5 mg of FT—amounting to administration to about 15-20% of the gland by volume). See, e.g., Shore, et al., “The potential for NX-1207 in benign prostatic hyperplasia: an update for clinicians,” Ther Adv. Chronic Dis., 2(6), pp. 377-383 (2011). In accordance with the embodiments described herein, and in light of the unexpected discovery that FT preserves prostatic nerve, stromal, vascular, and connective tissue, and urethral musculature, FT can be administered in significantly greater amounts than previously thought. In certain embodiments, FT can be administered, alone or in combination with another active agent, in an amount of from about 3.5 mg to about 350 mg, for an average weight male (about 86 kg—converts to about 0.04 to about 4 mg/kg of body weight), or from about 4.0 mg to about 250 mg, or from about 5.0 mg to about 150 mg, or from about 10.0 mg to 350 mg, or any value in between these ranges. In other embodiments, the same dosage of FT as previously administered (2.5 mg—12-20% by volume of the gland), or smaller dosages, can be administered to multiple locations in the prostate during the same procedure, or in the same or different locations at different time intervals ranging from one day to one week, repeatedly if needed for up to about 8 weeks, to increase the overall dosage within the above ranges, and treat substantially the entire prostate gland. The inventor also found in studies with beagle dogs treated with FT 0.28-1.6 mg/kg body weight that the prostate weights were consistently reduced after Intraprostatic single injection FT treatment (mean FT treated n=8 prostate weight 4.36 g, mean 3.4% of body weight; vs mean controls n=9, 8.96 g, 6.4% of body weight).

The following examples are provided to illustrate the present embodiments. It should be understood, however, that the embodiments are not to be limited to the specific conditions or details described in these examples. Throughout the specification, any and all references to a publicly available document, including a U.S. patent, are specifically incorporated by reference. In particular, the embodiments expressly incorporate by reference the examples contained in U.S. Pat. Nos. 6,924,266; 7,241,738; 7,317,077; 7,408,021; 7,745,572; 8,067,378; 8,293,703; 8,569,446; and 8,716,247, and U.S. Patent Application Publication Nos. 2017/0360885; 2017/0020957; 2016/0361380; and 2016/0215031, each of which reveal that certain peptides specified therein are effective agents for causing cell death in vivo in normal rodent muscle tissue, subcutaneous connective tissue, dermis and other tissue.

### EXAMPLES

Experiments were carried out at different times over a period of 5 years and therefore the number of rats per group while comparable was not strictly uniform. All protocols were done in accordance with applicable regulations, and carried out by individuals with training in animal handling and with anesthetic and other techniques to ensure painless procedural technique, and humane treatment of animals at all times.

### Example One

Two month old Sprague-Dawley rats (n=268) weighing 200 to 300 g, housed in groups of 2-5 per cage at room temperature (24-26° C.), with standard unrestricted diet and water, were anesthetized with ether and underwent open laparoscopic injections, using sterile precautions and sterile techniques, without antibiotics, of 0.3 mL of FT 0.1-2.0 mg/mL in phosphate buffered saline (PBS) pH 7.4, administered by #26 gauge sterile needles attached to sterile syringes. The animals received a “whole gland” injection in an amount roughly 20 times the amount previously administered to humans. Control animals (n=103) were injected with 0.5 mL solutions of (1) PBS vehicle alone; (2) HCl in water pH 3.0-5.0; (3) inactive synthetic peptides (n=8) in PBS pH 7.4; or (4) no injection. The rats were daily observed and painlessly sacrificed after post-treatment intervals of 24 hours to 12 months under either anesthesia.

Sub-groups of rats received repeated injections (2×-8×, on a once weekly basis) (Table 1). Postmortem examinations were limited to prostate (full independent toxicological studies in rats, rabbits, and dogs were independently carried out in separate studies not reported here, which have shown no toxic effects of FT).

Prostate glands were removed, bisected, and immersed in 10% formalin solution, and subsequently embedded in paraffin, sectioned, and stained with (1) hematoxylin-eosin (H&E); (2) Bielschowsky silver method for nerve fibers; and (3) immunohistochemical TUNEL staining. TUNEL (Terminal deoxynucleotidyl transferase (dUTP) nick end labeling) detects DNA fragmentation by labeling the 3′-hydroxyl termini in the double-stand DNA breaks generated during apoptosis. Prostate cell lines (PC3 and LNCAP) were treated with FT 0.001 or 0.25 mg/mL in 6, 24, and 48 well plates and harvested and pelleted at 0, 12, 24, and 48 hours. Electron microscopy was performed on the sectioned pellets (Analytical Biological Services, Wilmington, Del.; and Paragon BioServices, Baltimore, Md.). The same treated cell lines under the same conditions were also stained in vitro after treatment using Annexin V immunofluorescence methods and viewed under ultraviolet light, and cell loss in vitro was assessed quantitatively (Multitox-Fluor, Promega). Annexin V binds to phosphatidylserine which is a marker for apoptosis when externalized on the outer leaflet of the plasma membrane.

Apoptosis was evaluated microscopically in H&E stained sections, including rats sacrificed after 24, 48, and 72 hours, 4-8 days, and 1, 3, 6, and 12 months. All sections from FT treated rats were examined by two separate observers who tabulated extent of atrophy and apoptosis, nerve presence or absence, and nerve histological normality or abnormality, in each section. TUNEL staining was assessed in 21 animals (72 hours; 7 days, post-treatment). Prostate volume (calculated by approximation to a sphere using the mean of 8 perpendicular diameters (2 per section at 90 degrees; on four sections) and calculations of 4/3 π(D/2)³) were assessed in all animals and all controls. Tangentially cut blocks and sections were excluded from measurements.

A summary of animal groups according to treatment (concentration of treatment compound, frequency of treatment, interval post-treatment to sacrifice), and microscopically derived volume measurements, is shown in Table 1.

TABLE 1
Prostate Volume in FT Treated Rats and Controls¹

| | | Volume | | | Volume |
|---|---|---|---|---|---|
| | | mm³- | | | mm³-Mean |
| Group | N | Mean (SD) | Group | N | (SD) |
| Vehicle 24 hr | 2 | 566.4 (58.6) | FT 1 mg/mL 6 mos | 8 | 986.2 (560.9)⁷ |
| Vehicle 48 hr | 4 | 664.9 (475.8) | FT 1 mg/mL 12 mos | 4 | 450.3 (129.0)⁸ |
| Vehicle 72 hr | 8 | 621.6 (266.6) | FT 2 mg/mL 7 days | 5 | 251.7 (24.3)⁹ |
| Vehicle 96 hr | 5 | 392.6 (117.0) | FT 2 mg/mL 3 mos | 4 | 616.8 (148.7)¹⁰ |
| Vehicle 5 days | 2 | 1182.9 (453.9) | FT 2 mg/mL 6 mos | 4 | 776.3 (453.7)¹¹ |
| Vehicle 7 days | 11 | 560.5 (205.9) | FT 2 mg/mL 12 mos | 6 | 754.9 (483.3)¹² |
| Vehicle 8 days | 4 | 400.2 (99.7) | FT 5 mg/mL 7 days | 4 | 422.9 (33.2) |
| Vehicle 1 mo. | 2 | 1313.0 (406.7) | FT 1 mg/mL weekly X2 | 4 | 509.2 (200.3) |
| | | | wk, 3 mos | | |
| Vehicle 3 mos. | 2 | 966.3 (83.6) | FT 1 mg/mL weekly X2 | 4 | 497.3 (150.3) |
| | | | wk, 12 mos | | |
| Control Vehicle 12 mos | 1 | 1291.7 | FT 2 mg/mL weekly X2 | 4 | 651.9 (183.9) |
| | | | wk, 3 mos | | |
| No Injection 24 hr | 5 | 444.8 (74.0) | FT 2 mg/mL weekly X2 | 4 | 1401.1 (439.9) |
| | | | wk, 6 mos | | |
| No Injection 72 hr | 4 | 338.1 (144.5) | FT 2 mg/mL weekly X2 | 4 | 574.9 (264.1) |
| | | | wk, 12 mos | | |
| No Injection 5 days | 4 | 327.6 (112.1) | FT 1 mg/mL daily X3 | 12 | 240.0 (51.0) |
| | | | days, 3 days | | |
| No Injection 3 mos | 3 | 899.3 (174.7) | FT 1 mg/mL weekly X4 | 4 | 536.9 (87.7) |
| | | | wk, 3 mos | | |
| No Injection 6 mos | 3 | 1809.7 (130.6) | FT 1 mg/mL weekly X4 | 5 | 705.4 (246.2) |
| | | | wk, 6 mos | | |
| HCl pH 3.0-5.0, 24 hr-10 days | 12 | 859.7 (278.1) | FT 1 mg/mL weekly X4 | 8 | 745.9 (316.9) |
| | | | wk, 12 mos | | |
| Inactive n = 8 different | 8 | 550.6 (76.8) | FT 2 mg/mL weekly | 4 | 643.7 (270.3) |
| peptides 5-10 days | | | X4wk, 3 mos | | |
| Vehicle weekly X2, 12 mos | 2 | 1720.9 (606.9) | FT 2 mg/mL weekly X4 | 4 | 551.6 (246.8) |
| | | | wk, 6 mos | | |
| Vehicle weekly X4, 12 mos | 2 | 1099.5 (271.9) | FT 2 mg/mL weekly X4 | 6 | 770.5 (222.4) |
| | | | wk. 12 mos | | |
| Vehicle weekly X8, 12 mos | 2 | 1030.3 (174.0) | FT 1 mg/mL weekly X8 | 4 | 663.5 (70.9) |
| | | | wk, 3 mos | | |
| FT 1 mg/mL 24 hr | 20 | 387.0 (117.5)² | FT 1 mg/mL weekly X8 | 5 | 734.5 (203.3) |
| | | | wk, 6 mos | | |
| FT 1 mg/mL 48 hr | 16 | 226.4 (66.9)³ | FT 1 mg/mL weekly X8 | 3 | 738.0 (153.5) |
| | | | wk, 12 mos | | |
| FT 1 mg/mL 72 hr | 54 | 287.9 (104.7)⁴ | FT 2 mg/mL weekly X8 | 3 | 728.4 (116.2) |
| | | | wk, 3 mos | | |
| FT 1 mg/mL 7 days | 46 | 303.0 (102.7)⁵ | FT 2 mg/mL weekly X8 | 4 | 897.6 (353.6) |
| | | | wk, 6 mos | | |
| FT 1 mg/mL 3 mos | 4 | 674.2 (195.0)⁶ | FT 2 mg/mL weekly X8 | 8 | 805.7 (223.7) |
| | | | wk, 12 mos | | |
| All controls (all frequencies, | 92 | 661.4 (368.4) | All FT (all frequencies, | 268 | 476.8 (310.3)¹³ |
| all time intervals) | | | all time intervals) | | |
| All controls (≤7 days) | 64 | 587.5 (292.8) | All FT (≤7 days) | 157 | 297.3 (106.8)¹⁴ |
| All repeat controls 12 mos | 7 | 1264.9 (421.4) | All repeat FT 12 mos | 90 | 658.1 (317.1)¹⁵ |

¹Single administration unless noted
²Control Vehicle PBS pH 7.4
³p < .05 vs vehicle alone
⁴p = .0012 vs vehicle alone
⁵p < .0001 vs vehicle alone
⁶p < .0001 vs vehicle alone
⁷p = .0374 vs no injection
⁸p = .005 vs repeated injections
⁹p = .0055 vs vehicle alone
¹⁰p = .0405 vs vehicle alone
¹¹p = .013 vs no injection
¹²p = .0667 vs repeated vehicle injections
¹³p < .0001 vs all controls
¹⁴p < .0001 vs all controls ≤ 7 days
¹⁵p < .0001 vs all repeat controls 12 mos

The mean volume of FT treated rats at all frequencies, concentrations and time intervals was 476.8 mm³ (SD 310.3), compared to mean volume of controls 717.3 mm³ (SD 402.4) (p<0.0001, CI −317.62 to −163.38). The mean volume of all FT treated rats at all concentrations <7 days post-treatment (n=157) was 297.3 mm³ (SD 106.9), compared to the mean volume of controls <7 days (n=64) 587.5 mm³ (SD 292.8) (p<0.0001, CI −343.15 to −237.31).

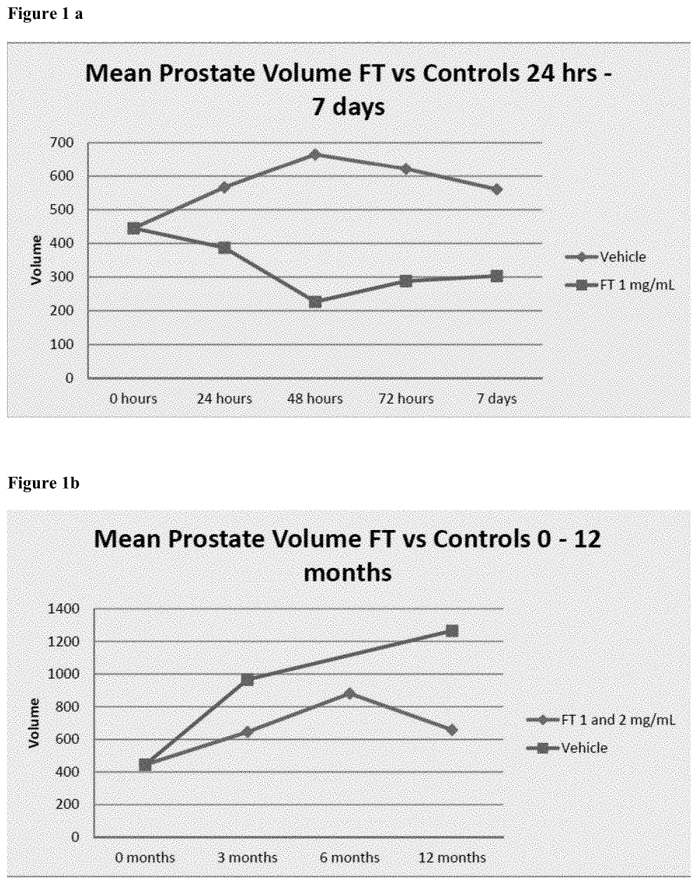

The mean volumes of FT 1 mg/ml treated rats vs vehicle alone (PBS) are shown graphically in FIGS. 1a and b. All adequately powered individual time point matched values were statistically significantly reduced in FT treated rats compared to the vehicle control injected animals. There were no apparent consistent differences in the reductions in volume found among single doses in the 0.5-5.0 mg/ml range, and there were no apparent differences between single and multiply FT injected rats in the 1 mg/mL or 2 mg/mL dosages.

In control sections there were occasional examples (6/92 control rats) with recognizable needle insertion tracts. Two (2/12) rats injected with HCl pH 3.0-5.0 had focal ischemic or hemorrhagic infarctions and necrosis involving <5% of the cross-sectional area. Other HCl pH 3.0-5.0 treated animals had microscopic foci (<2% of cross-sectional area) with focal necrosis. There were no other examples of injection induced hematoma >5% of the cross sectional area. All controls exhibited nerve presence. Controls did not show the histological features described below in FT treated rat prostate. Apoptotic figures in untreated controls were sparse (<1 per 100× field). Glandular epithelium showed no significant lasting changes in untreated controls or in any controls treated with vehicle alone or with inactive peptides in PBS. PBS injected prostates were swollen at time intervals <72 hours. At time intervals >7 days in saline injected rats prostates there was no further swelling detectable.

FT treated rats showed the following histological changes not found in controls: (1) apoptotic changes consisting of large areas with very prominent cellular changes of hyperchromatic pyknotic convoluted nuclei progressing to the appearance of smaller roundish broken nuclei and apoptotic bodies, with cellular dissolution with pallor, cell ghosts and cell disappearance at 24, 48, 72 hours, 1 week, and to a lesser extent in the ensuing weeks. At 6 months and one year, apoptotic changes were infrequent or no longer seen; (2) TUNEL positivity: dark brown immunoperoxidase TUNEL staining was seen in the areas of the apoptotic changes described above in 1. (3) normal appearing nerves at all time points including 6 months and 1 year; and (4) atrophy consisting of significantly reduced overall prostatic volume. Histologically, glandular epithelium was initially disrupted, and then progressively sloughed, and gradually disappeared. After 6 months-1 year, there was near-complete to complete loss of glandular epithelium throughout the prostate. Stromal connective tissue remained, and nerves and blood vessels were intact at all time intervals. Administration of FT therefore leads to apoptosis in mammalian prostate glandular epithelium and to widespread but selective gland epithelial cell loss and atrophy.

Ultrastructural changes found in vitro after 24-48 hours consisted of: (1) nuclear changes (hyperconvoluted electron dense nuclei with prominent invaginations and foldings); (2) nuclear membrane disruptions and eventually prominent nuclear blebs; (3) organellar disruptions with vesicular swelling and disruption; and (4) progressive cell disruption, fragmentation, and disappearance into debris. In vitro Annexin V positivity was demonstrated in prostate cell lines. Untreated controls and control wells treated with medium or with PBS vehicle were negative.

Quantitative measures of RNA showed depletion in FT treated prostate cell lines after 24 hours. In vitro quantitative measures of cell death and loss showed significant cell depletion with the 0.25 mg/mL FT dose compared to the 0.001 mg/mL FT dosage (Table 2). There were no statistical differences seen in vivo in rat prostate volumes after injections of dosages in the range of 0.5-5.0 mg/mL. There were also no consistently found significant changes overall after repeat injections, compared to single injections.

The studies exemplified herein demonstrate that FT leads to apoptosis in rat prostate glandular epithelium and to widespread but selective gland epithelial cell loss and atrophy. The reduction in prostate volume in the studies reported here is in the range of 33 to 50% compared to controls. The dosage in the studies reported here involved a volume infusion approximately equal to the volume of the prostate allowing for the FT to reach all or nearly all of the gland acinar epithelial cell populations. The rat prostate is highly cellular compared to the human BPH gland, the latter being up to 50% stromal in structure. Furthermore, the human BPH prostate weighs up to 70-100 g or more and the FT volume (10 mL dose) per prostate volume dosage in humans is proportionally smaller compared to the rat per prostate volume experimental dosage.

Gland-specific molecular ablation of overgrown prostatic glands in the transition zone in the prostate with nerve, stromal, vascular, and connective tissue, and urethral musculature sparing is a novel mechanism of action for a prostate therapeutic which has important benefits. The prostate gland performs vital male reproductive functions and is situated in intimate proximity to many important pelvic structures (urethra, bladder, rectum, seminal vesicles). Non-specific ablation in some percentage of patients inevitably leads to irreparable damage to important pelvic structures with resultant functional deficits. A review of known ablative devices and agents and their collateral damage toxicities suggests that, in general, prostatic nerve, stromal, vascular, and connective tissue damage commonly leads to sexual deficit (ejaculatory dysfunction, impotence, loss of libido); urethral damage commonly leads to retrograde ejaculation and/or strictures; and rectal or bladder damage can result in incontinence, fistulae, strictures, and/or dysfunction. The specificity of FT avoids the large spectrum of adverse events from non-specific ablation-related damage to other structures.

The extensive well known toxic effects of non-specific ablation are well documented in the device studies literature and device labels for high energy transduction ablation techniques (laser; needle ablations; microwave; cryotherapies; high intensity ultrasound), radiation (external beam; brachytherapy seeds); and in the literature for non-specific abrasives (carbolic acid; alcohol, etc). In the afore-mentioned, and other methods, there is non-specific ablation and inevitable permanent damage to some degree of delicate adjacent structures. Systemically administered chemotherapies are effective against rapidly growing cancerous tissues with side effects on other vulnerable tissues with high basal turnover rate or with receptors in common with the chemotherapy, whereby traditional chemotherapies are usually toxic to other tissues to some variable extent. For example, 5-alpha reductase inhibitors (5-ARIs) are testosterone pathway blockers that reduce prostate size by reducing individual prostate glandular cellular volume. 5-ARI induced cellular shrinkage is reversible and is not an ablation per se. 5ARIs do not ablate prostate cells or any adjacent cells; however 5 ARIs have many unwanted side effects on other tissues attributable to testosterone pathway imbalances (such as gynecomastia, impotence, loss of libido, and possible risk of higher grade prostate cancers).

As demonstrated herein, FT administration consistently leads to significant and selective prostate glandular epithelial apoptotic cell loss and gland shrinkage, in the absence of discemable damage to adjacent and surrounding tissues including nerve, stromal, vascular, and connective tissue, and urethral musculature, and other important structures. The selective nature of FT permits administration of larger doses than previously administered, and administration to substantially the entire gland, resulting in complete or near-complete reversal of the benign overgrowth, and negating the need for subsequent treatment.

1. A method of selectively destroying gland overgrowth comprising administering Fexapotide Triflutate to a patient in need thereof in an amount sufficient to treat a substantial portion of the prostate gland; and selectively destroying gland overgrowth, while substantially preserving nerve, stromal, vascular, and connective tissue, and urethral musculature in intimate structural proximity to the loci of administration.

2. The method of claim 1, wherein the method comprises administration of Fexapotide Triflutate as claimed in claim 1 and a carrier.

3. The method of claim 1, wherein the method comprises administration of Fexapotide Triflutate within the range of from about 0.04 to about 4 mg per kg of mammal body weight.

4. The method of claim 3, wherein the method comprises administration of Fexapotide Triflutate within the range of from about 0.05 to about 3 mg per kg of mammal body weight.

5. The method of claim 3, wherein the method comprises administration of Fexapotide Triflutate within the range of from about 0.06 to about 1.7 mg per kg of mammal body weight.

6. The method of claim 3, wherein the method comprises administration of Fexapotide Triflutate within the range of from about 0.12 to about 4 mg per kg of mammal body weight.

7. The method of claim 1, wherein the method comprises administration of Fexapotide Triflutate more than once at different locations in the prostate gland.

8. The method of claim 1, wherein the method comprises administration of Fexapotide Triflutate in an amount of less than about 0.04 to about 4 mg per kg of mammal body weight once a week for a period of from two weeks to 8 weeks.

9. The method of claim 8, wherein the method comprises administration of Fexapotide Triflutate in an amount of less than about 0.04 to about 4 mg per kg of mammal body weight once a week for a period of from two weeks to 4 weeks.

10. The method of claim 1, wherein the method comprises administration of Fexapotide Triflutate in an amount of less than about 0.04 to about 4 mg per kg of mammal body weight once a month for a period of from two months to 6 months.

11. The method of claim 10, wherein the method comprises administration of Fexapotide Triflutate in an amount of less than about 0.04 to about 4 mg per kg of mammal body weight once a month for a period of from two months to 4 months.

12. The method of claim 1, wherein the method comprises administration of Fexapotide Triflutate more than once.

13. The method of claim 12, wherein the period of time between administration of the Fexapotide Triflutate is within the range of from about 2 months to about 10 years.

14. The method of claim 13, wherein the period of time between administration of the Fexapotide Triflutate is within the range of from about 8 months to about 4 years.

15. The method of claim 13, wherein the period of time between administration of the Fexapotide Triflutate is within the range of from about 1 year to about 2 years.

16. The method of claim 1, wherein Fexapotide Triflutate is administered by a method selected from the group consisting of orally, subcutaneously, intradermally, intranasally, intravenously, intramuscularly, intrathecally, intranasally, intratumorally, topically, transrectally, transperitoneally, and transdermally.

17. The method of claim 1, wherein the method shrinks the prostate gland volume by an amount within the range of from about 15% to about 75% per single dose.

18. The method of claim 5, wherein the method shrinks the prostate gland volume by an amount within the range of from about 33% to about 48%.
